SEQUENCE LISTING (3 sequences)

SEQ ID NO: 1 - protein, 770 aa, Caenorhabditis elegans
MSSDTGSEYLDEEIRGDELGPSIDDNALAAAASAARLPFDRPTDHELAFFPELWEHKTAVEVFLLLRNSTLATWQYNPLKECTALDVRNNVFPPFNSDLDLIQNIVHYLSRHGLINFGRYVRSTKISRFLVRDRRSVIVIGAGAAGISAATQLESFGFDVIVLEARNCIGGRIHSFKSKSGEIMETGGDTLRKIEDSPMATLLHQVNFEEHGVFDFTSVFVEGRPLNEEKIHLFLDHYKSAHGALNYQAHQCEHRDDQGSFISRQQAYENLLSMCERGTLIKYYNFCKSLETVARAREHHFNQMKQLRMTALMAENQLKKMEEEGNLEQDPVLRRSLKRDIATSLEKFEEVADAFETADNHWQRLNEHPQAKQYMHPGSEFATFNFMLGFEEYLVGAQLEKVQFSCDSMQNKENGVAARLTEGIAELLTQLSEKRKLDIRLKHRVLDIDYSGFEHVLLKVQRENGDIEEMKAAFVVSTLPIGVLKKTIIADERAPTFTPSLPDKKVEAIRNIGCGSVNKCILEFDRVFWTANGGRNQFVTVSPNIKTRGSMNIWSSVPGSKVLCTYIVGEEAMLELPDDVIIQNAMINLQKAFGNNCPRAPISAHITRWHDDELAFGSGAFMSLRTETTSFDDVMEPLKTSDGMSRVYFAGEHTCSSYTSTIQGAWMSGARAAADISNDHIGIGFVDISGTRGQRGDEEEELLIEVDIDGKIPEKDENEAVADIPNAPNAPNAQKPEEIPKIAEEIELVAEAEKAEKAEVQLEPLVPTVE

SEQ ID NO: 2 - DNA, 2313 bp, Caenorhabditis elegans
atgtcgtctgacacgggttccgagtatttggacgaagagatacgtggcgatgaattggggccgtctatagatgataatgctctcgccgctgccgccagcgccgcccgtcttccattcgaccgcccgactgatcacgagttggcattcttccccgagctctgggagcacaagacagccgtcgaggtgttcctgctgctcagaaattcgacgctcgccacgtggcaatacaatccactgaaagagtgcacagcgttggatgtcagaaataacgtttttccgccgttcaactcagatttagacttgattcagaacattgtgcactatctatcccgtcatggactaatcaatttcggtcgttacgtgagatccaccaaaatcagtcgttttctagtccgtgatcggcgttcagtgattgtaattggcgctggagcagctggaatttcagcagccacccaactggaatcattcggattcgatgtaatcgttttggaggcaaggaattgtattggtggacgtattcacagttttaaatcgaaaagtggagagattatggagactggaggtgatacgctgcgaaaaatcgaggattcaccgatggccacgttgttgcatcaggtaaacttcgaagagcacggagtattcgatttcacctcagtattcgtcgagggtcgtccattaaacgaagaaaagattcatttattcctagatcactataaatctgctcacggagccctaaactatcaagcccatcaatgtgagcatcgtgatgatcagggatcatttatttcccgacaacaagcctatgagaatctattaagtatgtgtgaacgtggaacacttatcaaatattataacttttgtaaatcactggaaaccgtcgcccgagcccgtgagcatcatttcaatcaaatgaaacagttgagaatgacagcattgatggctgaaaatcagctgaaaaaaatggaagaagaaggaaatttggagcaggatccggtgctcagaagatcattaaaacgggatattgcaacatcactcgagaaatttgaagaagtcgctgacgcttttgagacggctgataatcattggcaacggcttaatgagcatccacaggcgaaacaatatatgcatccgggatctgaattcgcgacttttaattttatgttgggatttgaggagtatttggttggagcacaattggaaaaagtgcaattctcgtgtgattcgatgcaaaataaggagaatggagtggcggcgagattaaccgaaggaattgccgaactgctaacacaattatcggaaaaacgaaaattggatattcgtctgaagcatcgtgttttggatattgattatagcggatttgagcatgttttactgaaagttcagcgagaaaatggagatattgaagaaatgaaagccgcttttgtagtatctacacttccaattggtgtacttaaaaaaacaattatcgccgacgagcgtgctccgacgttcactccctcactacccgataaaaaagtggaagcaattcgaaatattggctgtggaagtgtgaataagtgtattctcgaatttgatcgagttttttggacggcaaatggtggaagaaatcaatttgtgacggtttcaccgaatataaaaacacgtggctccatgaatatctggtcatcagtgcctggctcaaaagtactgtgtacctacattgtaggcgaagaggcaatgctcgagctacccgatgacgtcataattcaaaatgcgatgataaatcttcaaaaagcatttggaaataattgtccacgtgctccaatcagtgcacatattacacgttggcatgatgatgagttggcatttggatccggtgcatttatgagcttaaggactgaaactacaagctttgatgacgtcatggaacccctcaaaacctccgatggaatgtcacgggtctattttgctggagaacacacgtgttccagctatacatcgacgattcaaggagcatggatgtctggggcgagagccgccgccgacatttccaacgatcatattggcatcggctttgtggatataagtggaacacgtggtcaacgcggtgatgaagaagaagagcttctcattgaagtggatattgatggaaaaattccggagaaggatgaaaatgaggctgtagctgatattccaaatgctccaaatgctccaaatgctcaaaaacccgaagaaatcccgaaaatcgccgaagaaattgaactcgtggctgaagctgaaaaggctgaaaaagctgaagtccagctggagccactagtgccaacagttgaatag

SEQ ID NO: 3 - DNA, 11863 bp, Caenorhabditis elegans
atgtcgtctgacacgggttccgagtatttggacgaagagatacgtggcgatgaattggggccgtctatagatgataatggtgaggaaatcctggaaaaagtgcagaaaattggataaaaatggcggtggtagaggctcgagtgtagtttgtagtgtgagcctcaaccaagttttcagcgaaaattcacgattttcagcagaaaaatcacagttttgcgctataataacaccaaatataagaaaaaaccattgagttttccatatttttctgctaaaaaagctttaaaattcaactttcaaattggttgaggctagaaatgttgtagtttgtagtgtgtgagcctcaaccagtttttcatccatttttaaggcaaaaataggcaattttacattaaaaattcacgattttcagtagaaaaatcgcatctttcgctattaatcgttaaaaacaaaaaaaattttctaaagttttcgcacagaaacaccaaatttaaaatgaaaatcaatacttggttgaggctagaaatgttgtagtttgtagtgtgtgagcctcaaccaataagttgtgcgatttacattgagaaaatctgaaaagcacagtcaaaaatgagaatttttggtagaaaacagcattttttccatttaaattcggaaaaatctcgaaaatgaccttgggtgaggctgagagaagtgtagtttgtagtctctgagcctcaaccaatttcttttttttttcttcgaatttaatataattttccctgtaattttttaaattaaagtgtcaaaaattccaggaaaaaggtgtagtttgtagtctaatttccccatttttttttcagctctcgccgctgccgccagcgccgcccgtcttccattcgaccgcccgactgatcacgagttggcattcttccccgagctctgggagcacaagacagccgtcgaggtgttcctgctgctcagaaattcgacgctcgccacgtggcaatacaatccactgaaagagtgcacagcgttggatgtcagaaataacgtttttccgccgttcaactcagatttagacttgattcaggtgtggaaattaggaaaatcgtcgattttgcacctaaaactttaaaaaaatcgagaaaatagatgaaaaactgcagttttttgtcgaaacatccgatttttgtcttgggtgaggctcagacactacaaactacaacatttatagcctcaacctttggttgaggctgaaacgtttgtagtttttagtctgcgagcctcaaccaaagttgattgtctgatttttcagatatttcatggatttttcccgaaaaatacaaatttttaagcaaaaattagaatggaaattcccaaaaatcggttaaaaacgccaatattcatagagttcagactacaaactacaaaacaattaaattttattgaaaaatgaaaaatttaaaatgaaatttcaaaattaaaaggcgaaattatatcaaaataaccaaaaacatttttgaattttcaacaaaaacctggttttttgaagaatttcaatttttttcaccgaaaaatctaattaaaaactacaaaatgaaaaaaaatttgttttttttgttcgtgaaaaattatattctataaattttaaaattataagtattaaaaaaataacgtttttccgccgttcaactcggatttggacttgattcaggtgtgaaaattaggattttcactgattttgcatctaaaaatcatgaaaaactccagaaaatcgattaaaaaaccgcagttttcagcgaaaaatgggtgaaaaatccgatttttgtcttgggtgaggctcagacactacaaactacaatttgcgctgagcctcaaccaatcaatttttgcttttttttagaatgaaaattggattttatattttattattctgatttgagatttaaaaataaaaatttaagaattttgaatttttttaaacagaaaatcggaattaaatgatcaaattttttgaaattttcgggcgaaaagtgattgagtgctggaaaatagttaaacttttatcaaaataactgaaaaatttgaattttcaatcatgaaaatggcaaattcagggattttgatgaaatttttatcgaaaaaatcgaattttcacctgaaaatttattgaaaatgcttatttttttgcgaattttgacaaaaaaaaaccgaaaaaaattcctttccgaatatttttttacagaaaatcggaatgaaattttcaaaattttgaaattttcgggcgaaaagtgattgagtgctggaaagtagttgaaaattatattttttggctatttttatagaaatttaaaaaaaaggattattgaaaaaatcgaattttctcctgaaaatttattgaaaatgcttatttttttgcgaattttgacaaaaaaaaaccgaaaaaaattcctttccgaatatttttttacagaaaatcggaatgaaagtttgagaaaattgcgaaataactaacaaaaaaaattgaattttcaaaaaaaaactggttttttttttttgaagaatttcaaattttttcactgaaaaatcaaataaaatgttttttgggaaaattgcaaaatttcattttaaaaaaatcgaattttcaactgaaagtttattgaaaatcctatttttttgcaaaaaatgaggttttttcaagaatttttgataaattactttccaaattttgtagtttgtagtttcaattttcagaacattgtgcactatctatcccgtcatggactaatcaatttcggtcgttacgtgagatccaccaaaatcagtcgttttctagtccgtgatcggcgttcagtgattgtaattggcgctggagcagctggaatttcagcagccacccaactggaatcattcggattcgatgtaatcgttttggaggcaaggaattgtattggtggacgtattcacagttttaaatcgaaaagtggagagattatggagactggaggtgatacgctgcgaaaaatcgaggattcaccgatggccacgttgttgcatcaggtttgtgcggaaaatcggtggaaattgcggaaaagtcgaggaaaattgattaaaaattggagattttatcaaaaatccgatttttgtatcaaaaacttgtgaaaaacctatgaaaaactgcagatttcggcgaaaaatacctgaaaaacggcaaatttgtagtagaaatatcattttttgtttaaaaaaattgtgaacatcgatcaaaaacggcatatttcagcgaaaaataccggaaaaacgacggaatttaatcaatttcgttttttggttgaggctcacactacaaactacaatcgttcttagcctcaagcattgttctggttgaggctgaaacgttcgtagtatgtagtctgggcctcaaccaaagtggattgaccaatttttcagaaatttcaataaaaaggaaaacattttccgtaatgtttatcgaattttattgagaaaattgattaaaaaactgcagattttagcaaaaatactcgaaaaatgtatttctccaaaacatctatgaaaaaatgcaatttattagcgaaaaaactcgaaaaacgacaaattttttggcaaaaattcaatttttgctgaaaaaaatttagaaaattgattgaaaaactgcagatttcaccgaaaaaatcccgaaaaacgacgaaatttaatcaaaatgacgattttcgttttggtggaggctcacactacaaactacaatcgttcttagcctcaaccattgttctggttgaggctgaagcgtttgtagtttgtagtctgagcctcaaccaaagttgatcaacgtattttccagaaatttcatggaattttcttgaaaaagacaaattttccgtaactttattgaaaaaatcgaaaaaaaaacgatttttgaacgaaaaatcatgttttcaaaaaaaattggttgaggctcaggctacaaactacactttttaatcctcaaccagtataaaagtaggttaaacattgtagtttgtagtctgagttggaaaatttgcaaattttactcaactctaaaaactacagtacctgtaactttgtagtttgtagtcagaaatttaccactccgcgtgatatatctttcattacaaactacaaatctaaagtatttgtggtttgtagtgagagctcacactacaaactacacaaaacattcaattttttgatatcgaaaacgaaaaagtgaaacattttttattaaataagtgttaaatttgacaaaattaattttttttttccgactttttttttggatttttcaataaaacaaaaatttcaggtaaacttcgaagagcacggagtattcgatttcacctcagtattcgtcgagggtcgtccattaaacgaagaaaagattcatttattcctagatcactataaatctgctcacggagccctaaactatcaagcccatcaatgtgagcatcgtgatgatcagggatcatttatttcccgacaacaagcctatgagaatctattaagtatgtgtgaacgtggaacacttatcaaatattataacttttgtaaatcactggaaaccgtcgcccgagcccgtgagcatcatttcaatcaaatgaaacagttgagaatgacagcattgatggctgaaaatcagctgaaaaaaatggaagaagaaggaaatttggagcaggatccggtgctcagaagatcattaaaacgggatattgcaacatcactcgagaaatttgaagaagtcgctgacgcttttgagacggctgataatcattggcaacggcttaatgagcatccacaggcgaaacaatatatgcatccgggatctgaattcgcgacttttaattttatgttgggatttgaggagtatttggttggagcacaattggaaaaagtgcaattctcgtgtgattcgatgcaaaataaggagaatggagtggcggcgagatgttagtgatttgaaaagagcaaaatttgatggaaaatttggaattttttcgttgaagaatttgaaagtttaaggaaaatgggaaaatttaaattttggacttttttttgacaaaaaattggcaaaattttataaaagtgcgaaaaaatcgaaaattttcgatttttcgaaaaatcgaacgagaagaaaattcaatggaaaacatttgaaatttcatgaagaaacctgaaaattgtgacgaattttcgatttttaggcaaaaaaaatgatttcctattttttttcactttcgaaaaaaaaactgaaattttgtcaaacaaacttaattcaatttttttaaagaatttttctgccaaaaattggattctctatttattttcgtgttaacgcatttgaaattcgagaaaaaattggaaattttcaattttggaattttttggcgaaaaaaaattccgcaaaaattgataaaattgcagaaaaaacggaaatttttagcgaaaaatagcttttcctgaaaattgaaaaattcacgtgtattttttgattttttgaatttttttcctaataaagtcgtaaaaatctgtgaaaattcacgaaaatctttcaaattttcggataattcagttcaaaacgctcgaaaatttgaataataatccaaaatccacgtggatttattgattttttgaatgatttttggattttggatagaaaatgtcaaaaatcgtcaaaatactaactagaacagtgataattgaaatttttagactatacaggcttaatccaatgaaatataatcatcaataaaattacagtaaccgaaggaattgccgaactgctaacacaattatcggaaaaacgaaaattggatattcgtctgaagcatcgtgttttggatattgattatagcggatttgagcatgttttactgaaagttcagcgagaaaatggagatattgaagaaatgaaagccgcttttgtagtatctacacttccaattggtgtacttaaaaagtacgtgccatttttcgagaaaaaaattgattcgaaaaaaaaacgaatttagcgtagtcgttttagaagcactgttaaaaaaattgcagaaaaaaattgaaaaataaccgaaaatcgcggggttttttgccgaaaacgctgaatttttcaggaaaaaaacgaaattttaaaaaataagtttatgccgaaaaatcagtaaaaaattgaatttttaacttaaattaacgccaaaaactaaaaaaaaaaccacatttttccaagaaaattgcgaaattttcactgaaaaaactcaaaactagaggaattcattcaattaggtgattaaaatagatatttgcctacaacattctgaaaattcgaatttttatcgctgaaaaatggggaaatgcggatttttccaaaaaaaaacgaaattttcagagtaaaaactagctatttggctttaaaatttgaaaaattgagtttttgttttttttccttcaaaaaattgagctgaaaatgaaaaaaacacatttttccagaaaaaagtagcaaaattttcattgaaaaaactcaaaaactagagaaattctgtacatttttcagattaaaatcgatatttggctacaaaattctaaaaaaaaaattgaatttttatcacaaaaagtacaggaaaatgtttaaaactaagagaacttgaattttattgtttttttcatctttttaatcaataaaatgcaagttctcttaagttttatacattttcgtgtattttttgtgataaaaattcaatttttttttttagaattttgtagccaaatatcgattttaatctgaaaaatgtacagaatttctctatttttgatttttttcaatgaaaattttgctcgtttcgactcaatttaactcaaattttcaaattttccaaatagctattttaagtctaaaaattcgaaaatctccgtttttcaccggaaaaatccgaaaaaatatgcatttttaagatcaaagatcaattttttagtagtaaaaaattcgactacaccacctttaaataaaaaataacacattttccagaacaattatcgccgacgagcgtgctccgacgttcactccctcactacccgataaaaaagtggaagcaattcgaaatattggctgtggaagtgtgaataagtgtattctcgaatttgatcgagttttttggacggcaaatggtggaagaaatcaatttgtgacggtttcaccgaatatgtgagcggttttttcggactacaaactataaaacgtactggttgaggctcaaagactacaaactacaaatttctgaggctcaccaagttttctgtcgttttttaagtttttccgaacgaatttttccctgaaaaactaggtttttgaacaaaatttctgttgtttttttcgggtttctattcgatttttaaaccaaatcttgtcagttcaaatttgattgaggcgtagactacaaactacactttttctgttagcctcaaccatttttcgcagaattttgcagtatttttttttttttgaaaaatcctaattttcgtttaaaaaatcaagatcttggcccgaaaatagcgaaaaaaacgtcaaatttgtattttaaaagaacataaattttggttgaggctcagagactacaaactacaattcatcctcagcctcaaccaattactagtgttgaaaatgtcacatttttagttacaacgttgggttttgaccgttttctcctatttttcgatgacaaaatttaagatttagcaagaaaaatctttaaggaaactgaaattcgctcgaaaatagcaaaaaaatttcaaattttcgaagaaaagcttttcactattttggttgaggctcatagactacaaactacaatttttgttagcctcaaccatagcgaaattattggaattaaaaaaaaatatttcgttaaaaaatttgtttttttaaagagtgacgaatcacactacaaactacacaattctaggaaaaaccgaatttttttccgataaaaaattttttgaaatcaggtttttgactacaaactacaaaaaaaaaaagttatttttggctaaaaaatctgaaaaaacactttttttggtaataaaattttatattttcggtagtttgtagtgtgtacatttcctaaaattttcgataaaaatcaatttttttttgaaaaaaattttggagctcccacaaaactacaaactacaaaatgttaaaatttaatttttgaaaaaaaaattgtctgatttttagatttgtagtttgtagccttttgaaaatgaaatttcctacgaatttctcattaataacgtttattctaagaagctccgactacaaactacaatattttatttttaaaacaattggttgaggctcagagactacaaactacacaaatctatcagcctcacccataattttaaattttcattatatttggatgaggctcagagactacaaactacaaactacacaaaattccattttttctatgaagaaatcaatttttaatagttttgaaatacaaaattgaaaaaaaaaaattttaattaggctcccacaaaactacaaactacaaaaagtcacattattagatttgtagtttgcagtctctcattaataacatttattcctttaaaaaaaaacaagaattgtgactgaaaaataagctccgactacaaactacaatatgactatttggttgagttgcgcagactacaaactacaaactacacaaaattaaatttttttttccagaaaaacacgtggctccatgaatatctggtcatcagtgcctggctcaaaagtactgtgtacctacattgtaggcgaagaggcaatgctcgagctacccgatgacgtcataattcaaaatgcgatgataaatcttcaaaaagcatttggaaataattgtccacgtgctccaatcagtgcacatattacacgttggcatgatgatgagttggcatttggatccggtgcatttatgagcttaaggactgaaactacaagctttgatgacgtcatggaacccctcaaaacctccgatggaatgtcacgggtctattttgctggagaacacacgtgttccagctatacatcgacgattcaaggagcatggatgtctggggcgaggtacagacactacaaactacaaactacacttttttttttgctggctacaaactacataattgtggtggaagcttgtagttgtgaggcccagcgtgactacaaactacactttttgactggcgaaaattgcaatttcttactagggaatattgttaataggggaattgctaggaagacacaacaaactttaaacgacagatgaggtctttggctacaaaaatcattttttggaatctgctaaactctgccccaaagctgaaattttcggccttaaaaagtagtcctctttttgctcaaaatgccgtttttcgtgtgtagaagtggctacataaaagtttggcactgcagccgccgttcgaatggccgattggtgaactctcgtgctcattaatctaaaggtcacgggttcgttccccactaggtattaatttttttttttgaatttttcctctagcatgaaaaatgcacaaaaacacaacaaattgtcagttgaatctactctgctaaattttatgggtagagagatatgggcattagtaattaagtatgttaaagcttcacttcgattatttattggtacttttgaaacaactttgcagttgacaacttttttttattttacccgtcaacggagctggaagtctttgaaagtaggtccttttcgtgaaaaactctagagaaaacaacagtggttttctcctaaaaatgacatttgatcctccaaaatcgtagaaatatccaaactaatgctgttctttcaaaccaaacactttttgaaaaaattttgaaaaattatttttgaagaaaaatatgtgcatatcaggtgggagaaaagtgaaaatttatcaaaaaaagaaaacttccaaaaagtttccaaattacttttatgattaccaggaaccaaaaaatttattccgaagttaccaaaaaacggaagaaaccgtgtttttgtgcatttttcatgctagaggaaaaattcaaaaaaaaaattaatacctagtggggaacgaacccgtgacctttagattaatgagcacgagagttcaccaatcggccattcgaacggcggctgcagtgccaaacttttatgtagccacttctacacacgaaaaacggcattttgagcaaaaagaggactactttttaaggccgaaaatttcagctttggggcagagtttagcagattccaaaaaatgatttttgtagccaaagacctcatctgtcgtttaaagtttgttgtgtcttcctagcaattcccctattaacaatattccctagttagcacaaatccaatttatattccaacaaaaaaaattgagaaaatcgatgaaaacctgcagatttcagcgaaaagcatcggaaaacgacaaaatttaatcgaaaatccaatttttgttttaagtgaggctcacactacaaactacaacatttttagcctcaaccattgttgtttggttgaggctgaaaatttgtagtttgtagtccgggcctcaaccaatgttgattgtccaattttccagaaatttcatggaattttcttgaaaaagacaaattttctgtaactttattgaaaaaatcgaaaaaaaaaacgatttttgaacgaaaaatcacgttttcaaaaaaaatttggttgaggctcacactacaaactacaacatttttagcctcaaccattgttgtttgattgaggctgaaacgtttgtagtttgtagtctattgtgaaaatacctgttttctgaaataaaaacggtgctaaaaagacacgtggatttattttaaaaaatcgcagttttctgtagtttgtagtgtaattaagtctcacactacaaactacaatgttttataaaaattcggaaaaaaagttattaaaaaatttgaaaattgccttttttattgaaataaaaattttttacaaaatttccaacaaaaaatgattttctgtgaaaaaattgttcactacaaactacaaaatttcgagaaatcttgtaaaaatttgaaaaatgtgataaaattctcatattttgctttaaagtttttttttcttacaaataactttttttggaaaaaaatttgaaggttttcatcttttttgtagtttgtagtgtaattagttatactacaaactacaataatttgtaacgaattccaaaaatttcgaagaaacatttttttgttgtagtttgtagtgtaaattcgataatttttacaatatttccgaagaaaaagctctaaaaattgaggacacactacaaactacaagataaataaagttgtagtttgtagtctgagacccttttgaaaatatttataaaaaatacagaaagtcaccattttttaacccaaaattgaaaaatctgctatcttaaatttgttccaaaaaatattgtagtttgtagtctacctaaaaaaaactacaaactacattttcttctatttttcagagccgccgccgacatttccaacgatcatattggcatcggctttgtggatataagtggaacacgtggtcaacgcggtgatgaagaagaagagcttctcattgaagtggatattgatggaaaaattccggagaaggatgaaaatgaggctgtagctgatattccaaatgctccaaatgctccaaatgctcaaaaacccgaagaaatcccgaaaatcgccgaagaaattgaactcgtggctgaagctgaaaaggctgaaaaagctgaagtccagctggagccactagtgccaacagttgaatag

(19) United States
(12) Patent Application Publication
(10) Pub. No.: US 2005/0059586 A1
(43) Pub. Date: Mar. 17, 2005

(54) Identification of ses-3 and the uses of the same

(76) Inventors: Ralf Baumeister, Freiburg (DE); Bernard Lakowski, Paris (FR); Stefan Eimer, Paris (FR)

Correspondence Address: ROSS J. OEHLER;AVENTIS PHARMACEUTICALS INC., ROUTE 202-206, MAIL CODE: D303A, BRIDGEWATER, NJ, 08807, US

(21) Appl. No.: 10/481,581

(22) PCT Filed: Jun. 24, 2002
(86) PCT No.: PCT/EP02/06964

(30) Foreign Application Priority Data
Jun. 22, 2001 (DE) 101 30 247.9

(51) Int. Cl. C12Q 1/68; A61K 38/17; C07K 14/47; C07H 21/04

(52) U.S. Cl. 514/12; 435/6; 435/69.1; 435/320.1; 435/325; 530/350; 536/23.5

(57) ABSTRACT

The invention relates to isolated nucleic acid molecules coding for SES-3 proteins or muted SES-3 proteins, and vectors and transgenic organisms containing such nucleic acid molecules. The invention also relates to uses of such nucleic acid molecules or others which are functionally similar, for producing pharmaceuticals and for producing model organisms. The invention further relates to the corresponding SES-3 proteins and muted SES-3 proteins, and the antibodies induced thereby. Finally, the invention relates to the use of substances which increase the expression of human presenilin, for the treatment of Alzheimer's disease, in addition to said substances themselves and pharmaceutical compositions containing the same.

The present invention relates to isolated nucleic acid molecules which encode ses-3 or mutated ses-3, to vectors and transgenic organisms which contain these nucleic acid molecules, to uses of these nucleic acid molecules, or of functionally similar nucleic acid molecules, for producing pharmaceuticals and for generating model organisms, to the use of transgenic organisms in methods for identifying substances which alter ses-3 activity or, respectively, increase presenilin activity (e.g. that of sel-12 or hop-1), and to the corresponding SES-3 proteins and mutated SES-3 proteins as well as antibodies to which these proteins give rise. The invention furthermore relates to the use of substances which increase the expression of a human presenilin for treating Alzheimer's disease, and to these substances themselves and to pharmaceutical compositions which comprise these substances.

### BACKGROUND TO THE INVENTION

Aside from Parkinson's disease, Alzheimer's disease is that neuro-degenerative disease which is most well known. The characteristic feature of Alzheimer's disease is the development of neuronal protein aggregations, what are termed plaques, which are essentially composed of an insoluble peptide, of 4 kDa in size, termed amyloid beta-peptide (Aβ4). Investigations carried out in the last few years indicate that the formation of Aβ4 is causatively involved in the development of the disease. Dominant mutations in three genes give rise to familial forms of the disease (FAD, familial Alzheimer's disease). It has been found that one of the genes concerned encodes amyloid precursor protein (APP), which can be processed by three proteases, resulting in the formation of Aβ4, inter alia. FAD mutations in APP increase the quantity of Aβ42, a 42 amino acid-long variant of Aβ4, which is produced.

Molecular genetic investigations of families in which Alzheimer's disease has occurred have led to the identification of the human genes presenilin 1 and presenilin 2 (Rogaev et al. 1995, Nature 376, 775-778; Levy-Lahad, E. et al. 1995, Science 269: 973-977) which, when altered by particular mutations, are causatively involved in the onset of Alzheimer's disease and which also play a key role in the Notch signal transduction pathway during development of the disease. Presenilin proteins are located in the ER-Golgi and possess at least 6 transmembrane domains. It has been found that mutations in PS1 and PS2 influence the formation of Aβ4 and are involved in giving rise to Alzheimer's disease, presumably by means of the formation of amyloid deposits. Members of the presenilin family have been identified in the mouse, in Drosophila melanogaster, in Xenopus laevis and in the threadworm Caenorhabditis elegans, inter alia. Although mutated presenilins influence the processing of APP in humans, their natural function in humans is not known in detail; it has been postulated that they might function as an APP protease (gamma-secretase). Other investigations carried out on human cell cultures have indicated that presenilins play a role in the intracellular proteolysis of Notch-like receptors after they have been activated by ligand binding. It is assumed, therefore, that the presenilins are involved in processing at least two different membrane proteins (APP and Notch). At present, it is a controversial matter as to whether the presenilins themselves are the proteases in this processing or whether they are cofactors of these reactions or influence the proteolysis indirectly (Haass, C. et al. 1999, Science 286 (5441): 916-919).

Taking the abovementioned presenilin genes as the starting point, intensive research is being carried out to elucidate the molecular disease processes and to develop pharmaceuticals for treating and preventing Alzheimer's disease. Factors which influence the activity of the presenilin genes are being sought, in particular, particular importance being attached, in this connection, to the search for suppressors of the presenilin mutant phenotype. Suppressors can be mutations in other genes which eliminate the requirement for the presenilin function or they can be substances which modulate the activity of other genes, or of the presenilins, such that the presenilin mutant defect does not have any phenotypic consequences.

A nematode, in particular Caenorhabditis elegans, has frequently been employed as a preferred model organism in these investigations. The advantage of a nematode model, in particular of the C. elegans model, lies, in particular, in its suitability for a high-throughput method (HTS; high-throughput screening), the possibility of faster genetic analysis due to a shorter generation time (2-3 days) and detailed knowledge of the molecular and functional properties of the nervous system in C. elegans. Since C. elegans can be kept on microtiter plates, it is possible to use this test system to test, by means of HTS, 10 000 or more substances on the living worm over a short period of time.

Three presenilin genes, designated sel-12, hop-1 and spe-4, have thus far been identified in the threadworm C. elegans. Spe-4 is the most divergent member of the family. Mutations in spe-4 lead to a defect in cytoplasmic partitioning during spermatogenesis (L'Hernault, S. W. et al. 1992, J Cell Biol 119: 55-68). While mutations in hop-1 do not have any visible phenotype, they lead, in combination with mutations in sel-12, to a synthetically lethal phenotype (Li, X. et al. 1997, Proc Natl Acad Sci USA 94: 12204-12209). Sel-12 is the gene which most strongly resembles human PS1 and PS2 and is also the best-studied presenilin gene in C. elegans. It has been found that certain sel-12 mutants exhibit an egg-laying defect and also morphological changes in vulva development (Levitan, D. et al. 1995, Nature 377: 3514). Sel-12 is 50% identical with human presenilins, and the egg-laying defect in sel-12 mutants can be offset by transgenically expressing PS1 or PS2 from the sel-12 promoter (Baumeister et al., 1997, Genes and Function 1: 149-159). This demonstrates that PS1 and PS2 exhibit functional homology with sel-12. Sel-12 mutations were initially identified as suppressors of the multivulva phenotype of a gain-of-function mutant in the lin-12 and glp-1 Notch receptor gene. Notch receptors control determination of cell fate in many multicellular organisms. In addition, it has been shown that the phenotype of sel-12 mutants resembles that of weak function-loss mutants of lin-12. In summary, it can be stated that presenilins play an important role in Notch signal transduction in a large number of organisms, including mammals.

### SUMMARY OF THE INVENTION

The present inventors have now found, surprisingly, that certain mutations in another gene, i.e. the C. elegans ses-3 gene, which was first identified by the inventors, are able to suppress a defect, in particular the egg-laying defect of a sel-12 mutant. These mutations are characterized by the fact that they alter, in particular reduce, the expression or activity of ses-3. This means that particular mutations in the ses-3 gene lead, for example, to the egg-laying defect, which is caused by certain sel-12 mutations, being eliminated and the double mutants (sel-12; ses-3) once again exhibiting a normal wild-type phenotype. More detailed investigations have shown that these mutations in the ses-3 gene lead, in the sel-12 mutants, to hop-1 being activated, i.e. the hop-1 presenilin protein takes over the function of the defective sel-12 presenilin protein (FIG. 1).

The present inventors have located the novel ses-3 gene on the C. elegans genome and sequenced it.

Consequently, the present invention initially relates to an isolated nucleic acid molecule which encodes SES-3 protein and to other nucleic acid molecules which encode mutated SES-3 proteins.

In addition to this, the invention relates to a vector which comprises these isolated nucleic acid molecules, or fragments thereof.

The invention furthermore relates to the SES-3 proteins and mutated SES-3 proteins, or fragments thereof, which are encoded by the nucleic acid molecules and to antibodies which are generated using these proteins.

In addition, the invention relates to a transgenic, nonhuman organism which comprises an isolated nucleic acid molecule which encodes SES-3 protein or mutated SES-3 protein. In particular, the invention relates to transgenic C. elegans organisms which exhibit an ses-3 allele which decreases the activity of ses-3 and, respectively, increases the activity of sel-12 or hop-1. In this connection, transgenic organisms are understood as being those organisms in which the genome has been altered by mutation, and which exhibit a mutated ses-3 gene as a result, or from which the ses-3 gene has been removed.

Finally, the invention relates to uses of these nucleic acid molecules for producing a pharmaceutical directed against Alzheimer's disease and for generating model systems for investigating this disease still further.

In particular, the invention relates to the use of transgenic C. elegans in a method for identifying and/or characterizing substances which reduce the activity of ses-3 or, respectively, increase the activity of sel-12 or hop-1.

The invention furthermore also relates to the use of homologous human genes, homologous Drosophila melanogaster genes or genes which exhibit functional similarity to ses-3 for producing a pharmaceutical which is directed against Alzheimer's disease and for generating model systems for investigating this disease still further.

Finally, the invention also relates to the use of substances which increase the expression of human presenilin 1 or 2 for treating Alzheimer's disease and to these substances themselves and to pharmaceutical compositions which comprise these substances.

### DETAILED DESCRIPTION OF THE INVENTION

The present invention is based on the fact that it has been possible to identify a novel gene, ses-3, in. C. elegans, with this gene interacting with the C. elegans presenilin genes sel-12 and hop-1 and thereby playing an important role in the development of Alzheimer's disease.

Thus, various mutations in the ses-3 gene, including complete deletion of the ses-3 gene as well, resulted in certain altered phenotypes which were elicited by sel-12 mutations being suppressed. The suppression of these sel-12 defects in ses-3 mutants is achieved by the latter markedly derepressing expression of hop-1 and by the HOP-1 protein presumably being able to take over the function of the defective SEL-12 protein.

The ses-3 gene which has been identified encodes a protein which contains an FAD binding site between amino acid 137 and amino acid 264 and an amino oxidase motif between amino acid 264 and amino acid 677 (FIG. 2). The SES-3 protein exhibits phylogenetic relatedness to the human protein KIAA0601 and to the Drosophila melanogaster protein ALT1; in addition computer searches (BLAST search program) identified a gene for another, similar C. elegans protein T08D10.2. The degree of homology is shown in FIGS. 3 and 4.

Consequently, the present invention provides an isolated nucleic acid molecule which encodes SES-3 protein.

The invention furthermore encompasses isolated nucleic acid molecules which encode mutated SES-3 proteins. In particular, the invention provides isolated nucleic acid molecules which encode mutated SES-3 proteins.

In addition to this, other aspects of the invention relate to isolated nucleic acid molecules, in particular DNA, such as cDNA or genomic DNA, molecules, but also RNA molecules, for example, which encode a C. elegans SES-3 protein having the amino acid sequence given in SEQ ID NO: 1, in particular those which exhibit a nucleic acid sequence given in SEQ ID NO: 2.

The invention also encompasses isolated nucleic acid molecules whose nucleotide sequences exhibit at least 75%, in particular at least 80%, especially at least 85%, preferably at least 90%, particularly preferably at least 95% and most preferably at least 98%, sequence similarity to the abovementioned nucleic acid molecules, in particular those which hybridize with the abovementioned nucleic acid molecules under stringent conditions. Such a hybridization preferably takes place under conditions of low stringency; in another embodiment, it also takes place under conditions of high stringency. In the context of this description, conditions of low stringency are understood as meaning a hybridization in 3×SSC at from room temperature to 65° C. and conditions of high stringency are understood as meaning a hybridization in 0.1×SSC at 68° C. SSC is the abbreviation for a 0.15 M sodium chloride, 0.015 M trisodium citrate buffer.

The nucleic acid molecules according to the invention having nucleic acid sequence similarity to the nucleic acid sequence given in SEQ ID NO: 2 also comprise, in particular, allelic variants of the given nucleic acid sequence, which variants include, in particular, the above-mentioned mutations.

The invention also extends to the nucleic acid molecules which in each case have a nucleic acid sequence which is complementary to the nucleic acid sequences described above.

In the present context, the term “isolated nucleic acid molecule” refers to nucleic acid molecules which are present in a form in which they are essentially purified from the main quantity of nucleic acid molecules of differing nature derived from the starting cells or producing cells. However, preparations of isolated nucleic acid molecules according to the invention can perfectly well contain other constituents such as salts, substances from the medium or residual constituents of the producing cells, such as various proteins.

The invention also encompasses vectors which contain a nucleic acid molecule according to the invention which encodes SES-3 protein or mutated SES-3 proteins. The invention also includes vectors which contain a fragment of a nucleic acid molecule according to the invention. In this connection, the nucleic acid molecule is linked to a promoter which causes the nucleic acid molecule to be expressed in the organism which is used for the expression. In this connection, one of the known C. elegans promoters (http://chinook.uoregon.edu/promoters.html) is used, in particular, for expressing in C. elegans.

The vector can, for example, be a plasmid, a cosmid, a bacmid, a virus or a bacteriophage.

Another aspect relates to the polypeptides or proteins which are encoded by the described nucleic acid molecules, in particular C. elegans SES-3 protein having the amino acid sequence given in SEQ ID NO: 1 and the abovementioned mutated SES-3 proteins and also polypeptides or proteins which are derived therefrom by means of substitution, modification, deletion, insertion or addition of amino acids and which exhibit at least 75%, in particular at least 80%, especially at least 85%, preferably at least 90%, particularly preferably at least 95% and most preferably at least 98% sequence similarity to the abovementioned amino acid sequences. The invention also encompasses fusion proteins which contain the poly-peptides, proteins or protein fragments according to the invention fused to a protein of a different nature or to a protein segment of a different nature, e.g. a marker protein or indicator protein. The invention also includes fragments of the abovementioned proteins or polypeptides.

The antibodies which can be obtained using these proteins, and which can be monoclonal or polyclonal, are likewise encompassed by the invention.

The invention also extends to transgenic organisms, in particular microorganisms, e.g. bacteria, viruses, protozoa, fungi and yeasts, algae, plants or animals, and also parts, e.g. cells, and propagation materials, e.g. seed, from these transgenic organisms, which comprise a recombinant nucleic acid sequence, where appropriate integrated into a chromosome or else extrachromosomally, which contains, as a transgene, a nucleic acid molecule according to the invention which encodes a SES-3 protein or mutated SES-3 protein, or fragments thereof. Under a preferred aspect, the transgenic organisms will also express the polypeptide or protein encoded by the abovementioned transgene, i.e. C. elegans SES-3 protein or a polypeptide or protein which is derived therefrom.

According to one aspect of the invention, transgenic C. elegans organisms exhibit an ses-3 allele which decreases, amplifies or eliminates the activity of ses-3. The invention also includes those transgenic C. elegans organisms from which the ses-3 gene has been completely removed. In particular, the present invention relates to transgenic C. elegans organisms which exhibit an ses-3 allele which offsets the egg-laying defect phenotype (Egl) in sel-12 mutants. In addition, the present invention relates to transgenic C. elegans organisms which exhibit an ses-3 allele which increases the activity of hop-1 or sel-12.

The invention also encompasses the use of the nucleic acid molecule according to the invention for producing a pharmaceutical for treating neurological, in particular neurodegenerative, diseases, particularly preferably Alzheimer's disease, or for generating a model organism for the further investigation and elucidation of neurological, in particular neurodegenerative, diseases.

The abovementioned pharmaceuticals can be obtained, for example, by employing a nucleic acid molecule according to the invention itself as a gene therapy agent or by using such a nucleic acid molecule to construct a model organism and formulating the substances which are found using this model organism into a pharmaceutical. For further details in this regard, the reader is referred, for example, to international application PCT/EP01/03214 belonging to the same applicant.

In particular, the invention relates to the use of transgenic C. elegans organisms, which contain a nucleic acid molecule according to the invention, in a method for identifying and/or characterizing substances which alter, in particular decrease or eliminate, the activity of ses-3. The invention furthermore relates to the use of transgenic C. elegans organisms, which contain a nucleic acid molecule according to the invention, in a method for identifying and/or characterizing substances which increase the activity of hop-1 or sel-12.

The invention furthermore relates to the use of these same transgenic C. elegans organisms in a method for identifying and/or characterizing substances which can be used as active compounds for treating and/or preventing Alzheimer's disease. In view of the analogy with human cells, and against the background of the demonstrated functional similarity of the Notch signal pathway and presenilin function in C. elegans and humans, it is assumed that the malfunction of the FAD presenilin mutants can be offset in an equivalent manner by mutating a gene which is homologous, or functionally equivalent, to ses-3 in humans. Such a homologous gene is, in particular, the gene KIAA0601. Within the context of the invention, it is expected, therefore, that a defect in a human ses-3 homolog, or a gene which is functionally equivalent, will lead to an increase in the expression of the presenilin gene which is not affected by the FAD mutation.

The invention therefore also relates to the use of substances which increase the expression of a human presenilin for treating Alzheimer's disease, and to these substances themselves. In particular, the substances according to the invention include those which activate human presenilin gene 1 or presenilin gene 2 and increase the expression of human presenilin protein 1 or human presenilin protein 2. The substances according to the invention also include those which, in C. elegans, lead to a change in ses-3 activity and/or increase sel-12 or hop-1 presenilin activity. The invention also encompasses pharmaceutical compositions which comprise these substances.

In an analogous manner, this invention can also be of benefit in the treatment of the sporadic cases of Alzheimer's disease which are age-dependent and not coupled to mutations. In this connection, it is expected that the decrease in expression will, by increasing presenilin activity, decrease the formation of Aβ4, in particular of Aβ42, and thereby chronologically delay the onset of the disease.

In addition, the invention also relates to the use of the homologous human gene (KIAA0601), and to the use of the homologous Drosophila melanogaster gene as shown in FIG. 4 (ALT1), and of functionally similar genes, in particular derived from C. elegans, for finding substances which can be used as active compounds for treating and/or preventing Alzheimer's disease. In particular, the invention relates to the use of these genes for finding substances which are able to increase the activity of human presenilins. The invention also relates to transgenic organisms which contain these homologous or functionally similar genes and to the use of these organisms, in particular C. elegans organisms, in a method for identifying and/or characterizing substances which increase hop-1 or sel-12 activity or decrease ses-3 activity.

### BRIEF DESCRIPTION OF THE FIGURES AND OF THE SEQUENCE LISTING

FIG. 1: Concentration of hop-1 in ses-3 alleles. Northern blot, hybridized with a hop-1-specific probe.

FIG. 2: Domain structure of SES-3 protein.

FIG. 3: Comparison of the amino acid sequence of SES-3 with that of homologous proteins.

FIG. 4: Phylogenetic relationship of SES-3, and homology.

FIG. 5: Detection of ses-3 mRNA in dependence on the different mutations. Northern blot.

FIG. 6: Genomic structure of the ses-3 region and depiction of the genomic DNA transgenes which complement ses-3 mutants.

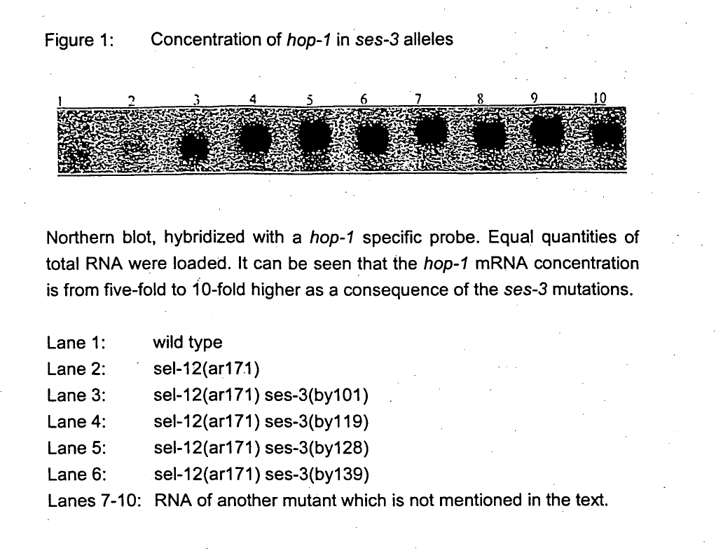

SEQ ID NO: 1: SES-3 amino acid sequence

SEQ ID NO: 2: ses-3 cDNA sequence

SEQ ID NO: 3: Genomic sequence of a subfragment of Y40B1B.6 which is able to complement ses-3 mutants. This fragment consequently contains all the sequences and promoter segments which are required for expressing ses-3.

The invention is explained in more detail below with the aid of examples.

Experimental Section

Isolating the ses-3 Mutants

In order to isolate, in C. elegans, suppressors of the egg-laying defect seen in sel-12 mutant animals, the sel-12 (ar171) mutation was crossed into a mutator strain mut-7(pk704). The resulting strain exhibits a temperature-dependent activation of all C. elegans transposons at 20° C. For the mutagenesis, sel-12(ar171) unc-1(e538); mut-7(pk704) animals were cultured at 20° C. on 3.5 cm NGM agar plates containing OP50. After each generation, a search was carried out for the appearance of animals which were able to lay eggs. From analyzing about 30 000 haploid genomes, two independent mutant animals were isolated which were able to lay eggs reproducibly. The egg-laying behavior of these animals was comparable to that of wild-type animals. One of these mutations was designated ses-3(by101).

The sel-12; ses-3 Phenotype

Mutations in ses-3 suppress the egg-laying defect of sel-12 mutants virtually completely. 95% of the sel-12(ar171); ses-3(by101) double-mutant animals exhibited an egg-laying behavior which was similar to that of the wild type whereas 5% of the animals exhibited defects in egg laying which correspond to the defects in the sel-12 single mutant. Mutations in ses-3 are able to suppress all known sel-12 mutations. It can be concluded from this that ses-3 does not act in an allele-specific manner. Sel-12(ar171); ses-3(by101) double-mutant animals lay between 180 and 280 eggs which lead to offspring; this is in contrast to the sel-12(ar171) single mutant, which produces an average of 60 offspring. The sel-12(ar171); ses-3(by101) double-mutant animals end their egg laying after about 2-3 days and do not die with a “bag-of-worms” phenotype, as is customary in the case of the sel-12 single mutation. As compared with wild-type animals, sel-12(ar171); ses-3(by101) double-mutant animals exhibit a growth behavior which is retarded by about one day per generation.

Cloning the ses-3 Gene Locus

Classic genetics was used to locate ses-3 as a recessive mutation on the right arm of chromosome I. Since the ses-3 mutation was produced by means of transposon mutagenesis, it was probable that the mutation was brought about by the insertion of a transposon into the coding region of the ses-3 gene. This results in what is termed a polymorphism as compared with the sel-12 single-mutant strain. This can [lacuna] after removing the remaining polymorphisms, generated by the mut-7 background, by means of outcrossing with the wild-type C. elegans strain N2. The transposons in the sel-12(ar171); ses-3(by101) strain which has been outcrossed in this way can be visualized by means of transposon display. By means of comparing with the wild-type sel-12 strain, it is possible to identify the transposons which are only present in the sel-12(ar171); ses-3(by101) double-mutant animals. Using this method, a Tc3 insertion was identified in exon 7 of the gene Y40B1B.6 which could not be separated from the ses-3 mutation by further outcrossing.

Transgene Rescue of ses-3

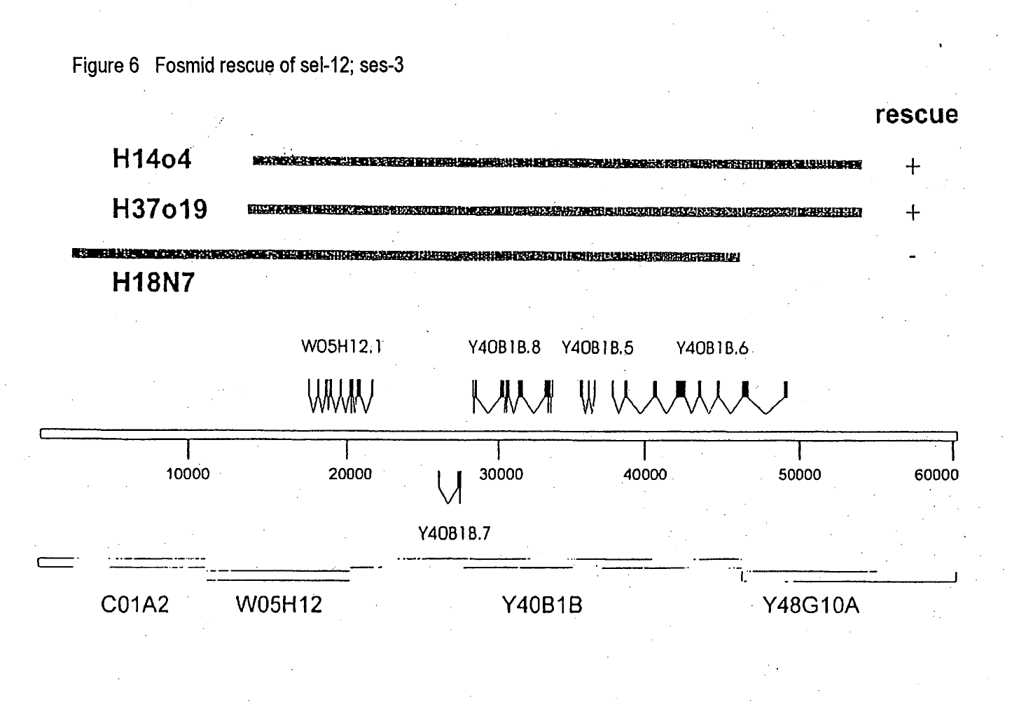

In order to rescue the ses-3 phenotype, the fosmids H14o4, H37o19 and H18N7, which contain the ses-3 genomic region, were isolated. In this connection, fosmids H1404 and H37o19 contain the complete genomic sequence of Y40B1B.6 whereas H18N7 only contains a part of the Y40B1B.6 genomic sequence which lacks exons 7 and 8. Transgenes containing individual examples of these fosmids were injected into the strain sel-12(ar171); ses-3(by101) and the antisuppressor activity of the transgenic animals was tested. All the clones were injected at a concentration of 50 ng of the given fosmid/μl, together with 100 ng of the dominant transformation marker pRF4 (Mello et al., 1991)/μl. The suppression of the egg-laying defect of sel-12-mutant animals by ses-3 was virtually completely offset (rescued) by the presence of the fosmids H14o4 and H37o19, whereas fosmid H18N7 was unable to rescue the ses-3-mediated suppression of sel-12 (see FIG. 6).

Structure of ses-3

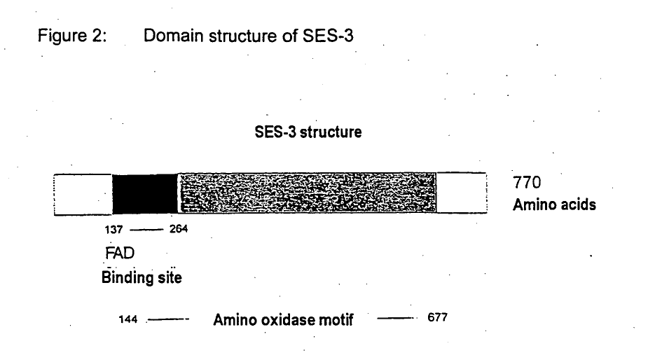

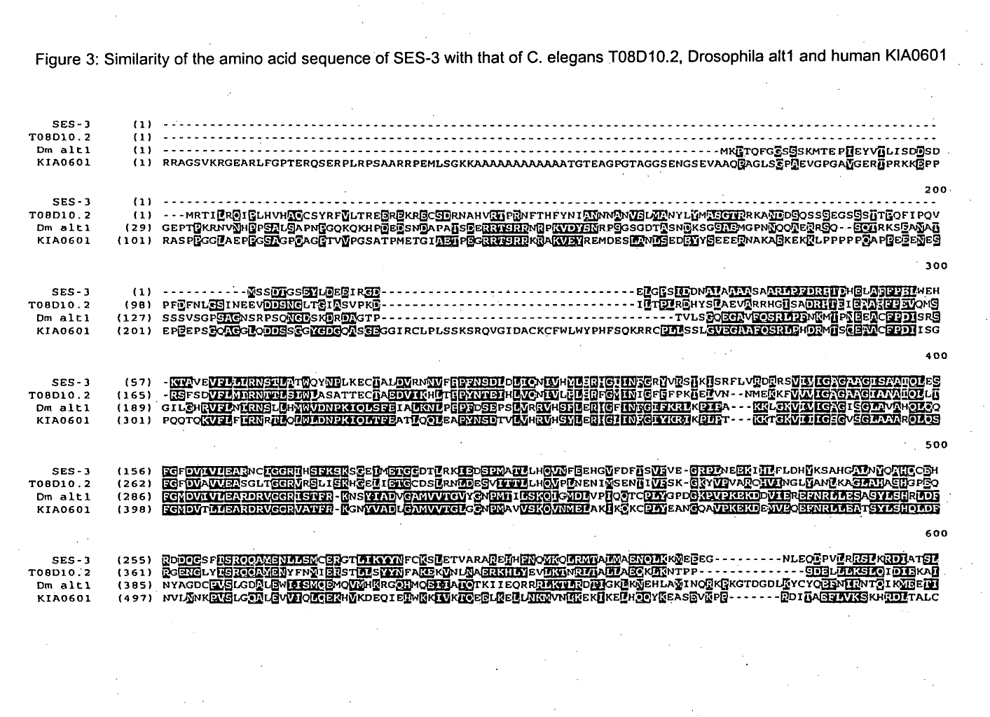

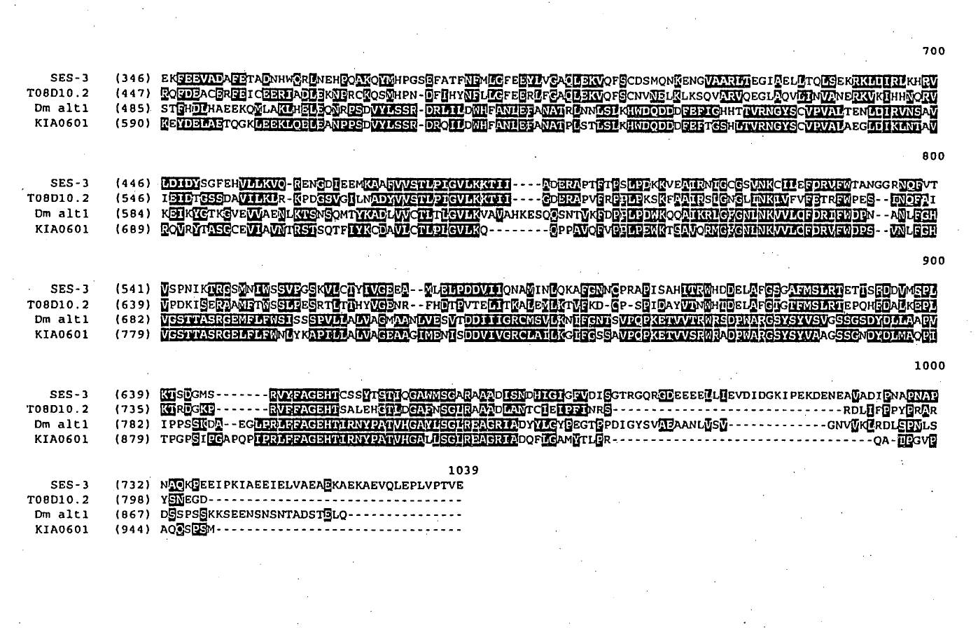

ses-3 (Y40B1B.6) is organized in 8 exons and forms an operon together with the gene Y40b1B.5 (see FIG. 6). The cDNA predicted for Y40B1 B.6 tallies with the cDNA of 2313 base pairs (SEQ ID NO: 2) which was determined for ses-3. Ses-3 encodes a protein of 770 amino acids (SEQ ID NO: 1). A motif search using Ses-3 showed that Ses-3 possesses an amino oxidase motif over the majority of its length and an FAD binding site at the N terminus (FIG. 2). Comparisons with sequences in the database showed homologies with another C. elegans gene, i.e. T08D10.2, and with a human EST KIAA0601, and a predicted Drosophila gene alt1 (FIG. 3).

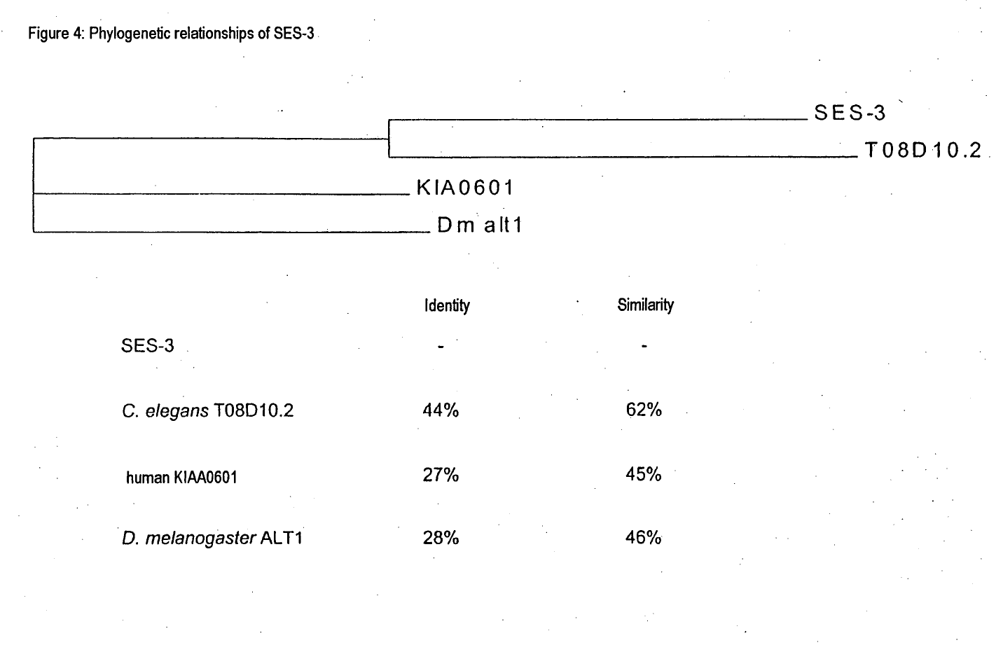

FIG. 4 shows how the genes are related to each other.

Expression of ses-3

A stage-specific Northern blot shows that ses-3 is expressed in all developmental stages.

Mutations in ses-3

by101:

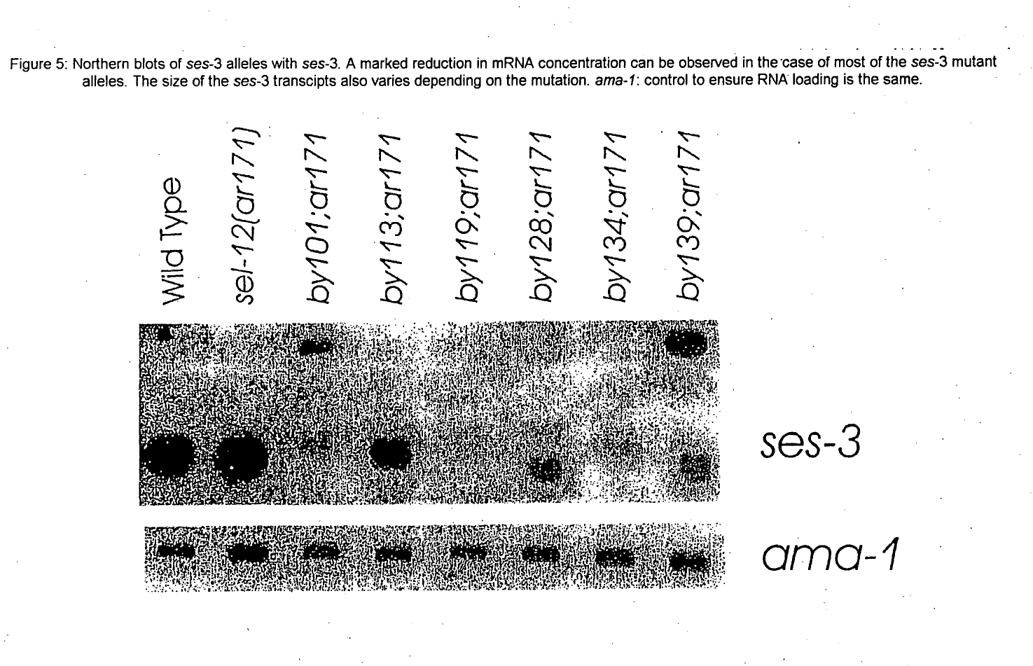

is a Tc3 insertion in exon 7 of Y40B1B.6 at position 1817 in the cDNA and affects expression of the mRNA (FIG. 5).

by113:

was isolated from an EMS mutagenesis and does not have any effect on expression of the mRNA (FIG. 5).

by119:

was isolated from an EMS mutagenesis and attenuates expression of the mRNA (FIG. 5).

by128:

was isolated from a UV/TMP mutagenesis and gives rise to attenuated expression of the mRNA and a rearrangement of the mRNA and of the genomic region (FIG. 5).

by134:

was isolated from a UV/TMP mutagenesis and attenuates expression of the mRNA (FIG. 5).

by139:

was isolated from a UV/TMP mutagenesis and gives rise to attenuated expression of the mRNA, a band which is reduced in size, the appearance of a second, larger band and a deletion in exon 8 from position 2015 to 2169 in the cDNA (FIG. 5).

Mechanism of the Suppression of sel-12 by ses-3

ses-3 mutations are unable to suppress the sterility of a sel-12; hop-1 ses-3. This demonstrates that hop-1 is required for suppressing sel-12. A Northern blot of the sel-12; ses-3 double mutants containing the ses-3 alleles by101, by119, by128 and by139 shows that hop-1 expression is upregulated in the L1 stage relative to sel-12 and the wild-type strain N2. Since hop-1 is able to replace sel-12, hop-1 consequently replaces sel-12 in the early larval stages.

1-31. (Cancelled).

32. An isolated nucleic acid molecule that encodes SES-3 or a mutated form of SES-3.

33. An isolated nucleic acid as described in claim 32, wherein the mutated form of SES-3 is a protein having at least one of an FAD binding site and an amino oxidase motif.

34. An isolated nucleic acid as described in claim 32, wherein the mutated form is a protein having one or more alterations in activity.

35. An isolated nucleic acid molecule as described in claim 32, which is DNA, cDNA, genomic DNA, or RNA.

36. An isolated nucleic acid molecule as described in claim 32, wherein the SES-3 has an amino acid sequence identical to, or essentially the same as, SEQ ID NO: 1.

37. An isolated nucleic acid molecule as described in claim 32, comprising a nucleotide sequence of SEQ ID NO: 2, or a sequence that is complementary thereto.

38. An isolated nucleic acid molecule, comprising a nucleotide sequence having at least 75% sequence similarity to a nucleotide sequence as described in claim 37, or a sequence complementary thereto.

39. An isolated nucleic acid molecule that hybridizes with an isolated nucleic acid molecule as described in claim 37 under conditions of 0.1×SSC at 68 degrees centigrade.

40. A vector that comprises an isolated nucleic acid molecule or a fragment thereof as described in claim 32.

41. A vector as described in claim 40, which comprises a promoter for expression in a eukaryote cell.

42. A vector as described in claim 40, which is in the form of a plasmid, a cosmid, a bacmid, a virus or a bacteriophage.

43. An SES-3 protein comprising the amino acid sequence of SEQ ID NO: 1, or a fragment thereof.

44. A mutated SES-3 protein, or a fragment thereof.

45. An SES-3 protein as described in claim 43, comprising one or more additions, deletions, or alterations at one or more amino acid positions corresponding to one or more amino acids of SEQ ID NO: 1.

46. An antibody produced against an SES-3 protein as described in claim 43.

47. An antibody as described in claim 46, wherein the antibody is a monoclonal antibody or a polyclonal antibody.

48. A transgenic, nonhuman organism that comprises a nucleic acid molecule as described in claim 32.

49. A transgenic, nonhuman organism as described in claim 48, wherein the organism is C. elegans.

50. A transgenic C. elegans organism as described in claim 49, which expresses an SES-3 allele that decreases, amplifies or eliminates the activity of SES-3.

51. A transgenic C. elegans organism as described in claim 50, which expresses an SES-3 allele that offsets the egg-laying defect phenotype (EgI) in sel-12 C. elegans mutants.

52. A transgenic C. elegans organism as described in claim 50, which expresses an SES-3 allele that increases the activity of hop-1 or sel-12.

53. A transgenic organism that comprises a foreign SES-3 gene homologue.

54. A transgenic organism as described in claim 53, wherein the homologue is human gene KIAA0601 or Drosophila melanogaster ALT1.

55. A method of producing a pharmaceutically active substance useful for treating neurodegenerative disease, comprising expressing an isolated nucleic acid molecule as described in claim 32, or a functionally similar gene, to produce a gene product and then isolating the gene product.

56. A method for preparing a transgenic organism for investigating neurodegenerative disease, comprising incorporating an isolated nucleic acid molecule as described in claim 32 into an organism.

57. A method for discovering substances that alter the activity of SES-3, comprising monitoring SES-3 activity in the presence of a test substance, and identifying or characterizing an effect of the substance on the monitored activity.

58. The method of claim 57, wherein the SES-3 activity is monitored in a transgenic organism or transgenic cell.

59. The method of claim 58, wherein the transgenic organism is E. elegans.

60. A method for evaluation of substances that increase the activity of at least one of hop-1 and sel-12 in a transgenic cell or organism containing a nucleic acid molecule as described in claim 1, comprising monitoring the activity in the presence of a test substance, and identifying or characterizing an effect of the substance on the monitored activity.

61. The method of claim 29, wherein the organism is C. elegans.

62. A method for evaluation of substances that affect the expression of presenilin in a transgenic cell or organism containing a nucleic acid molecule as described in claim 1, comprising monitoring presenilin expression in the presence of a test substance, and identifying or characterizing an effect of the substance on the monitored expression.

63. A method for producing a medicament for treating Alzheimer's disease, comprising providing a substance identified by the method of claim 31, and formulating the substance into a pharmaceutical.

64. A substance that increases the expression of human presenilin I or presenilin 2, comprising a molecule discovered by the method of claim 62.

65. A pharmaceutical composition that comprises a substance as described in claim 64.
